(19) United States
(12) Patent Application Publication
(10) Pub. No.: US 2004/0142326 A1
(43) Pub. Date: Jul. 22, 2004

(54) Method and apparatus for deriving a reference sequence for expressing a group genome

(75) Inventors: Barry Robson, Bronxville, NY (US); Richard Mushlin, Ridgefield, CT (US)

Correspondence Address: Ryan, Mason & Lewis, LLP, Suite 205, 1300 Post Road, Fairfield, CT, 06430, US

(73) Assignee: International Business Machines Corporation, Armonk, NY (US)

(21) Appl. No.: 10/269,192

(22) Filed: Oct. 11, 2002

(51) Int. Cl. C12Q 1/68

(52) U.S. Cl. 435/6

(57) ABSTRACT

A computer-based method is provided for deriving a reference sequence for expressing a group genome. The method includes determining a probability of occurrence for a base value in the reference sequence based on base value occurrences in the group genome. The determined probability of occurrence is then inserted in the reference sequence. The probability of occurrence is preferably determined for a plurality of base values.

### FIELD OF THE INVENTION

The present invention relates to the electronic transmission of data and, more particularly, to a computer-based method for expressing a group genome.

### BACKGROUND OF THE INVENTION

Sequencing the human genome and other recent advances in the field of bioinformatics suggest that the medicine of the future will take advantage of genomic data. For example, researchers and health care providers anticipate the ability to design drugs or screen a variety of drugs based upon the drugs' ability to bind to a protein coded by a patient's gene sequence. In addition, the Internet is already widely used to obtain medical information. Medical data are among the most retrieved information over the Internet. With a projection of one billion individuals on the Internet by the year 2005, new challenges will be presented to efficiently transport such volumes of genomic data. Computers and the Internet are also being utilized more and more frequently for data mining of genomic sequences. This increased volume of transmissions involving genomic data will demand more efficient ways to forward genomic information and other information related thereto.

The transmission of the genomic data of a group is difficult because of the large amount of data present. Conventional methods of electronically transmitting genomic data are unnecessarily slow and more prone to errors and unauthorized access. Errors occurring in the transmission of genomic data can have dire consequences, especially if used in the treatment of a patient. Thus, there exists a need for an improved method of data transmission in expressing a group genome.

### SUMMARY OF THE INVENTION

The present invention provides solutions to the needs outlined above, and others, by providing improved group genome expression. Disclosed herein is a method for deriving a reference sequence for expressing a group genome. The method comprises the steps of determining a probability of occurrence for a base value in the reference sequence based on base value occurrences in the group genome; and inserting the determined probability of occurrence in the reference sequence.

The method further includes the step of determining the probability of occurrence for a plurality of base values in the reference sequence, and expressing it as a percentage of the base value occurrences in the group genome. The preferred base values are adenine, cytosine, guanine and thymine.

A more complete understanding of the present invention, as well as further features and advantages of the present invention, will be obtained by reference to the following detailed description and drawings.

### BRIEF DESCRIPTION OF THE DRAWINGS

FIG. 1 illustrates an exemplary genomic messaging system (GMS);

FIG. 2 is a block diagram of an exemplary hardware implementation of a GMS; and

FIG. 3 is a block diagram illustrating a method for deriving a reference sequence.

### DETAILED DESCRIPTION OF PREFERRED EMBODIMENTS

The present invention will be illustrated below in the context of an illustrative genomic messaging system (GMS). In the illustrative embodiment, the invention relates to the expression of DNA sequence data. However, it is to be understood that the present invention is not limited to such a particular application and can be applied to other data relating to a genome including, for example, RNA sequences.

The GMS relates to software in the emergent field of clinical bioinformatics, i.e., clinical genomics information technology (IT) concentrating on the specific genetic constitution of the patient, and its relationship to health and disease states. Clinical bioinformatics is distinct from conventional bioinformatics in that clinical bioinformatics concerns the genomics and the clinical record of the individual patient, as well as that of the collective patient population. Thus, there are not only medical research applications which could benefit from the invention, but also healthcare IT applications, such as those in the category of e-health.

The clinical application of genomics and bioinformatics requires special consideration for the privacy of the patient (see, e.g., George J. Annas, “A National Bill of Patients' Rights,” in “The Nation's Health,” 6th edition, eds. P. R. Lee & C. L. Estes, Jones and Bartlett Publishers, Inc., 2001), the safety of the patient and for the production of informed decisions by the patient and the physician. The federal Health Insurance Portability and Accountability Act (HIPPA) has been recently introduced to enforce the privacy of online medical data. According to HIPPA, one must now recognize and address the above concerns in transmitting, storing or manipulating patient genomic data.

Since the system of the invention may be involved in a variety of medical care scenarios, including emergency medical care, it has been designed to be minimally dependent on other systems. The messaging network can include direct communication between laptop computers or other portable devices, without a server, and even the exchange of floppy disks as the means of data transport. Basic tools for reading representations of the transmission can be built in and used, should all other interfaces fail.

Another advantage of the invention is that it can conform to clinical information technology standards recommended by the Health Level Seven organization (HL7). HL7 is a not-for-profit ANSI-Accredited Standards Developing Organization that provides standards for the exchange, management and integration of data that supports clinical patient care and healthcare services. For example, HL7 has proposed a Clinical Document Architecture (CDA), which is a specific embodiment of XML for medical applications. Although HL7 is the prominent standards body, aspects of these standards are still in a state of flux. For example, there are few if any recommendations from HL7 regarding genomic information.

A block diagram of an exemplary GMS 100 is shown in FIG. 1. The illustrative system 100 includes a genomic messaging module 110, a receiving module 120, a genomic sequence database 130 and, optionally, a clinical information database 140. Genomic messaging module 110 receives an input sequence from genomic sequence database 130 and, optionally, clinical data from clinical information database 140. Genomic messaging module 110 packages the input data to form an output data stream 150 which is transmitted to a receiving module 120.

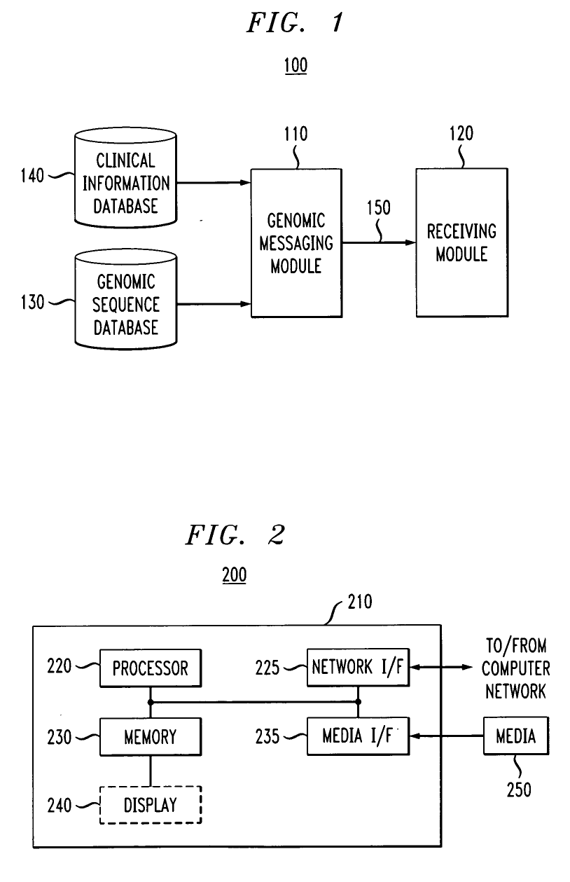

FIG. 2 is a block diagram of a system 200 for deriving a reference sequence for use in the expression of a group genome in accordance with one embodiment of the present invention. System 200 comprises a computer system 210 that interacts with a media 250. Computer system 210 comprises a processor 220, a network interface 225, a memory 230, a media interface 235 and an optional display 240. Network interface 225 allows computer system 210 to connect to a network, while media interfaces 235 allows computer system 210 to interact with media 250, such as a Digital Versatile Disk (DVD) or a hard drive.

As is known in the art, the methods and apparatus discussed herein may be distributed as an article of manufacture that itself comprises a computer-readable medium having computer-readable code means embodied thereon. The computer-readable program code means is operable, in conjunction with a computer system such as computer system 210, to carry out all or some of the steps to perform the methods or create the apparatuses discussed herein. The computer-readable code is configured to determine a probability of occurrence for a base value in the reference sequence based on base value occurrences in the group genome; and insert the determined probability of occurrence in the reference sequence. The computer-readable medium may be a recordable medium (e.g., floppy disks, hard drive, optical disks such as a DVD, or memory cards) or may be a transmission medium (e.g., a network comprising fiber-optics, the world-wide web, cables, or a wireless channel using time-division multiple access, code-division multiple access, or other radio-frequency channel). Any medium known or developed that can store information suitable for use with a computer system may be used. The computer-readable code means is any mechanism for allowing a computer to read instructions and data, such as magnetic variations on a magnetic medium or height variations on the surface of a compact disk.

Memory 230 configures the processor 220 to implement the methods, steps, and functions disclosed herein. The memory 230 could be distributed or local and the processor 220 could be distributed or singular. The memory 230 could be implemented as an electrical, magnetic or optical memory, or any combination of these or other types of storage devices. Moreover, the term “memory” should be construed broadly enough to encompass any information able to be read from or written to an address in the addressable space accessed by processor 220. With this definition, information on a network, accessible through network interface 225, is still within memory 230 because the processor 220 can retrieve the information from the network. It should be noted that each distributed processor that makes up processor 220 generally contains its own addressable memory space. It should also be noted that some or all of computer system 210 can be incorporated into an application-specific or general-use integrated circuit.

Optional video display 240 is any type of video display suitable for interacting with a human user of system 200. Generally, video display 240 is a computer monitor or other similar video display.

It is to be appreciated that, in an alternative embodiment, the invention may be implemented in a network-based implementation, such as, for example, the Internet. The network could alternatively be a private network and/or a local network. It is to be understood that the server may include more than one computer system. That is, one or more of the elements of FIG. 1 may reside on and be executed by their own computer system, e.g., with its own processor and memory. In an alternative configuration, the methodologies of the invention may be performed on a personal computer and output data transmitted directly to a receiving module, such as another personal computer, via a network without any server intervention. The output data can also be transferred without a network. For example, the output data can be transferred by simply downloading the data onto, e.g., a floppy disk, and uploading the data on a receiving module.

The GMS language (GMSL) is a novel “lingua franca” for representing a potentially broad assortment of clinical and genomic data, for secure and compact transmission using the GMS. The data may come from a variety of sources, in different formats, and be destined for use in a wide range of downstream applications. GMSL is optimized for the annotation of genomic data.

The primary functions of GMSL include:

retaining such content of the source clinical documents as are required, and combining patient DNA sequences or fragments;

allowing the expert to add annotation to the DNA and clinical data prior to its storage or transmission;

enabling addition of passwords and file protections;

providing tools for levels of reversible and irreversible “scrubbing” (anonymization) of the patient ID etc.;

preventing the addition of erroneous DNA and other lab data to the wrong patient record;

enabling various forms of compression and encryption at various levels, which can be supplemented by standard methods applied to the final file(s);

selecting methods of portrayal of the final information by the receiver, including the choice of what can be seen; and

allowing a special form of XML-compliant “staggered” bracketing to encode DNA and protein features which, unlike valid XML tags, can overlap;

GMSL, like many computer languages, recognizes two basic kinds of elements: instructions (commands) and data. Since GMS is optimized for handling potentially very large DNA or RNA sequences, the structures of these elements are designed to be compact.

A class of commands, relating to a byte mapping principle, allows four bases to be packed into a single byte to give the most compressed stream. This feature is useful for handling long DNA sequences uninterrupted by annotation. The tight packing continues until a special termination sequence of non-DNA characters is encountered. This compressed data can either be transmitted in the main stream, or read from separate files during the decoding process. Another type of command can be used to open or close a “bracket,” like parentheses, for grouping data together. These commands can be used to delineate a particular stretch of a genomic sequence for processing. Unlike parentheses, or markup tags, which can only be “nested,” e.g., {a[b(c)d]e}, GMS brackets can be crossed, e.g., {a[b(c}d)e]. This feature is important for genomic annotation because regions of interest often overlap. It also allows the same part of a sequence, or overlapping parts of sequences to be processed, e.g., annotated or qualified, in a plurality of ways at the same time.

In addition to these “mixed” commands, there are commands which are not associated with any particular portion of the genomic sequence, as well as commands which are associated with a number of bytes of genomic data. Command codes can be primarily informational. For example, a special command can indicate that a deletion or an insertion of a genomic base or a run of such bases, occurs at that point.

When sequences are experimentally unreliable at some location in the genomic sequence or it is experimentally unclear whether a particular nucleotide base is, for example, A or G, the sequence can be interrupted by commands indicating that one reliable fragment is ended and that the subsequent fragment has a level of uncertainty. Thus, the ability to keep track of multiple fragments is included within the GMS, including the ability to introduce comments. The GMS has the ability to keep count of the segments and, optionally, separate and annotate them in, for example, in the XML output.

A sample command phrase, or a group made up of several commands, can be as follows:

password;[&7aDfx/b{by shaman protect data];
xml;[<gms:{patient}_dna>\];index;and protein;
filename[template.gms{by shaman unlock data} ];read in dna
xml;[</gms:{patient}_dna>\];index;and protein;

Here the command “password” in the command phrase “password;[&7aDfx/b {by shaman protect data],” allows the incoming stream to be read and to be active from that point only if (a) the receiver has already entered a patient ID which encrypts to &7aDfx/b, and (b) if at that point the receiver enters another password, here “shaman.” Data item “filename;[template.gms{by shaman unlock data}]” allows the data of the file specified to be incorporated into the stream only if that password, here “shaman,” was the last entered, helping to ensure that the correct file is loaded and to ensure that the field has not been intercepted and falsely continued by a hostile agent. Another password command, with a different password requested, could follow the first password request.

A valuable DNA annotation command is of the example form:

(43

which forces the tag onto the final XML output file, e.g., <open feature=“whatever” type=“43” level=8/> depending on the bracket level. The command is used to annotate overlapping features, for example, DNA and protein features, which are impermissible to XML (in the sense that to XML <A><B></B></A>is XML-permissible, <A><B></A></B>is not).

Generic DATA statements encode specific or general classes of data which include, for example:

| | |
|---|---|
| data ;[........................./]; | |
| password ;[........................./]; | |
| filename;[........................./]; | |
| number ;[........................./]; | |
| xml;[........................../]; | (XML) |
| perl;[..........................{end of data} ] | (Perl applet executed on |
| | receipt) |
| h17;[.............................{end of data} ] | (HL7 messages) |
| dicom;[.........................{end of data} ] | (images) |
| protein ;[........................./]; | |
| squeeze dna;*.........................../] | (compress DNA to 4 characters |
| | per byte.) |

Alternative forms like “data;/ . . . / ” are possible. The terminating bracket “]” is optional and is actually a command to parity check the contents of the data statement on receipt. Within the fields “[ . . . ” can be inserted text permitted by “type.” Type restriction is currently weak, but backslash would be prohibited in certain types of data to avoid the fact that it is a permissible symbol in content.

A wide variety of commands in curly brackets (often referred to as French braces) can appear in these DATA fields, such as {xml symbols}, {define data}, {recall data}, {on password unlock data}, or carry variable names such as {locus} which are evaluated and macro-substituted into the data only on receipt.

The basic language can be used to make countless phrases out of the combinations, but there are relatively few complex commands formed. For example, the commands

filedata;[ {by shaman unlock data} ]
number;[15 base pairs\]
squeeze dna
*

AGCTTCAGAGCTGCT\

place a protective lock on the following data, requiring a password (in this example “shaman”) for access. The commands also compress 15 base pairs of DNA into four base pairs per byte, to the extent possible. Another example is:

name;[mary\];xml;[elizabeth {define data}]

xml;[<test>patient {identifier} has informal code name {mary}</test>\];index

which illustrates both the use of the use-defined variable “mary” and the system variable “identifier” (the current patient identifier) in writing specifically stated XML (the <test> tags and their content).

The genomic data input file (.gmd) contains the DNA sequences and the optional manual annotation. The DNA sequences are strings of bases. White space is ignored. The annotation is inserted using XML-style tags with a “gms” prefix, but the file is not an XML document.

“Cartridges” as used herein are replaceable program modules which transform input and output in various ways. They may be considered as mini “Expert Systems” in the sense that they script expertise, customizations and preferences. All input cartridges ultimately generate .gms files as the final and main input step. This file is converted to a binary .gmb file and stored or transmitted. Input cartridges include, for example, Legacy Conversion Cartridges, for conversion of legacy clinical and genomic data into GMS language.

When the .gmi file is a CDA document, as might be expected when retrieving data from a modern clinical repository, GMS needs to know how to convert the content, marked up with CDA tags, into the required canonical .gms form. This is accomplished using a GMS “cartridge.” In this scenario representing the first GMS cartridge application supporting automation, the expert optionally modifies a file obtained in CDA format to include additional annotation and structure. Again, the template mode described above is available to help guide this process so that the whole modified document remains CDA compliant. The resulting CDA document with added genomic features represents a “CDA Genomics Document.” Such a CDA document can now be automatically converted into GMSL. In addition to the legacy record conversion cartridge described above, automatic addition of genomic data is also contemplated by the invention so that the CDA Genomics Document is itself automatically generated from the initial CDA genomics-free file.

For example, genomic data can be merged using a gms: namespace prefix at the end of the CDA <body>, in its own CDA <section> as shown below using CDA structure:

```
<cda:clinical_document_header>
    .
    .<! --header structures per CDA-->
    .
</cda:clinical_document_header>
<cda:body>
    .
    .<! --clinical sections per CDA-->
    .
    <cda:section>
        <cda:caption>
            IBM Genomic Messaging System Data
        </cda:caption>
        <cda:paragraph>
            <cda:content>
                <cda:local_markup ignore=“markupr”>
                    <!--gms: tags go here-->
                </cda:local_markup>
            </cda:content>
        </cda:paragraph>
    </cda:section>
</cda:body>
```

More precisely, the cartridge looks first to see if the tags already exist in the document, in which case the cartridge will keep the tags. If the tags are missing, the cartridge will look for a <gms:body or <body tag (case-insensitively). If, however, there is no body tag, the cartridge will insert a <gms:body or <body tag (case-insensitively) before the last tag in the document. More information on GMS and the processing of data including a genomic sequence is discussed in U.S. patent application Ser. No. 10/185,657, filed Jun. 28, 2002, entitled “Genomic Messaging System,” incorporated herein by reference.

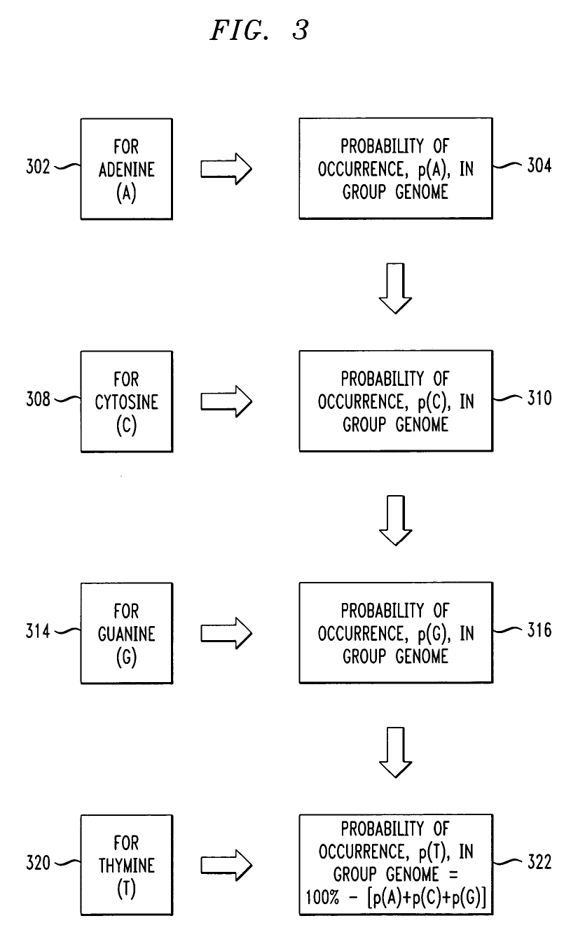

An exemplary method for deriving a reference sequence used in expressing a group genome is shown in FIG. 3. To derive the reference sequence, a probability of occurrence is determined for a base value. The base value represents a nucleotide base. Preferred nucleotide bases include, but are not limited to, the purines: adenine (A) and guanine (G), and the pyrimidines: cytosine (C) and thymine (T) or uracil (U) (i.e., uracil in RNA).

Preferably, the probability of occurrence 304, 310, 316 and 322 is determined for a plurality of base values, namely adenine (A) 302, cytosine (C) 308, guanine (G) 314 and thymine (T) 320, respectively. The probability of occurrence 304, 310, 316 and 322 represents the probability that one of adenine (A) 302, cytosine (C) 308, guanine (G) 314 or thymine (T) 320 occurs at a given locus in the reference sequence, based on the occurrences of adenine (A) 302, cytosine (C) 308, guanine (G) 314 and thymine (T) 320 in the group genome. The term locus may be defined as a specific position in a nucleotide sequence. The locus may be represented by a locus value. For example, the locus values one, two and three may be used to denote the first, second and third positions of a nucleotide sequence. The probability of occurrence for each base value reflects the occurrences of that base value in the corresponding locus of a plurality of sequences in the group genome. The term “group” is used to describe any population, sub-population, or grouping of individuals. Preferably, the group is a sub-population. Suitable sub-populations for use in the present invention may be defined by several parameters, including but not limited to, race, ethnic group, tribe, clan, family and sibling group. The methods of the present invention may be used to determine reference sequences for each sub-population considered to be a group. By grouping individuals into sub-populations, more universal genomic characteristics, such as pilot regions of a protein and intron regions of a gene, as well as more polymorphic protein characteristics such as glycosylation, are recognized.

In a preferred embodiment of the invention, the probability of occurrence 304, 310, 316 and 322 represents a percentage of the group genome that has the base value adenine (A) 302, cytosine (C) 308, guanine (G) 314 or thymine (T) 320 at corresponding loci. For example, if 50% of the group genome expresses the base value adenine at the fifth locus (i.e., represented by the locus value five), then the probability of occurrence, p(A), of adenine in the reference sequence at the fifth locus would also be 50%. Further, the probability of occurrence of any one of adenine (A) 302, cytosine (C) 308, guanine (G) 314 or thymine (T) 320 may be between 0% and 100%, for any given locus. Thus, in the instance where, e.g., the probability of occurrence, p(A), 304 is 100%, the probability of occurrence p(C) 310, p(G) 316 and p(T) 322 are each all 0%.

Preferably, the probability of occurrence is determined for at least three of adenine (A) 302, cytosine (C) 308, guanine (G) 314 and thymine (T) 320. Since, there are four possible base values that occur in a DNA sequence, then the probability of occurrence for a fourth base value may be determined once the probability of occurrence is determined for the other three base values. In a preferred embodiment, the probability of occurrence is consistently determined for adenine (A) 302, cytosine (C) 308 and guanine (G) 314 for each reference sequence, for each genome. Thus, the probability of occurrence for thymine (T) 320 may be determined as the difference of a 100% probability of occurrence less the sum of the probability of occurrence of adenine (A) 302, cytosine (C) 308 and guanine (G) 314.

The determined probability of occurrence 304, 310, 316 and 322 is then inserted into each corresponding locus in the reference sequence. An exemplary reference sequence may be depicted as follows:

. . . (40, 30, 10)(20, 20, 60)(50, 10, 40)(33, 33, 34)(90, 5, 5) . . .

If it is standardized that the probability of occurrence is determined in the order of adenine (A) 302, cytosine (C) 308, guanine (G) 314 and thymine (T) 320, then the probability of occurrence values present in the reference sequence above are clear. The three probability of occurrence values in each parentheses represent a percentage probability of occurrence for adenine (A) 302, cytosine (C) 308 and guanine (G) 314, in that order. The probability of occurrence for thymine (T) 320 can thus be determined from what is presented.

Additionally, a look-up table may be employed to determine the base value that corresponds to the probability of occurrence value. An exemplary look-up table might read:

| Position | Base Value |
|---|---|
| 1 | A |
| 2 | C |
| 3 | G |
| 4 | T |

Thus, in the table above, the first probability of occurrence value represents adenine, the second probability of occurrence value represents cytosine, the third probability of occurrence value represents guanine and the fourth probability of occurrence value represents thymine. Thus, for a set of values . . . (40, 30, 10) . . . , as above, the use of the look-up table would reveal:

| Position | Example | Base Value |
|---|---|---|
| 1 | 40 | A |
| 2 | 30 | C |
| 3 | 10 | G |

The fourth position representative of the base value T can be determined from the values displayed as being:

| Position | Example | Base Value |
|---|---|---|
| 4 | 20 | T |

In the instance wherein the probability of occurrence for any one base value is 100%, the probability of occurrence for each of the other three base values being 0%, that base value can be inserted in the reference sequence and no other probability of occurrence values need be represented. Further, following from the discussion above, the probability of occurrence 304, 310 and 316 may be inserted into the corresponding locus in the reference sequence. The probability of occurrence, p(T), may then be calculated as above.

It is to be understood that the teachings of the present invention, although described in terms of the expression of DNA nucleotide sequences, are also applicable to other sequence data, including but not limited to, RNA sequences. Thus, for deriving a reference RNA sequence, the nucleotide uracil would be present instead of thymine as described above.

### EXAMPLE

For a particular stretch of DNA, the sampling of a group, a population, shows the following sequences to be present, in the following percentages shown:

| | locus | | | | | | | | | | | | | | |
|---|---|---|---|---|---|---|---|---|---|---|---|---|---|---|---|
| | 1 | 2 | 3* | 4 | 5 | 6* | 7 | 8* | 9 | 10 | 11* | 12 | 13* | 14 | 15 |
| 50% | A | G | A | C | T | G | A | T | G | C | G | C | G | G | G |
| 30% | A | G | C | C | T | G | A | A | G | C | C | C | A | G | G |
| 10% | A | G | G | C | T | C | A | T | G | C | C | C | A | G | G |
| 10% | A | G | T | C | T | C | A | A | G | C | G | C | G | G | G |

*denotes a point of difference among individuals in the group.

Using the order: adenine (A), cytosine (C), guanine (G) and thymine (T) as the standard, the reference template for this population would be represented, according to the teachings of the present invention, as the following sequence:

locus

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| A | G | 50, 30, 10 | C | T | 0, 20, 80 | A | 40, 0, 0 | G | C | 0, 40, 60 | C | 40, 0, 60 | G | G |

Looking at locus 3, for example, it is shown that 50% of the population have adenine (A), 30% have cytosine (C), 10% have guanine (G) and the remaining (10%) have thymine (T).

Looking at locus 6, for comparison with locus 3, it is shown that none of the population have adenine (A), 20% have cytosine (C), 80% have guanine (G) and thus none of the population have thymine (T).

Although illustrative embodiments of the present invention have been described herein, it is to be understood that the invention is not limited to those precise embodiments, and that various other changes and modifications may be effected therein by one skilled in the art without departing from the scope or spirit of the invention. The following examples are provided to illustrate the scope and spirit of the present invention. Because these examples are given for illustrative purposes only, the invention embodied therein should not be limited thereto.

What is claimed is:

1. A method for deriving a reference sequence for expressing a group genome, comprising:
determining a probability of occurrence for a base value in the reference sequence based on base value occurrences in the group genome; and
inserting the determined probability of occurrence in the reference sequence.

2. The method of claim 1, wherein the group genome comprises a sub-population.

3. The method of claim 2, wherein the sub-population is identified by a parameter, including any one of race, ethnic group, tribe, clan, family and sibling group.

4. The method of claim 1, wherein the probability of occurrence is determined for a plurality of base values in the reference sequence.

5. The method of claim 1, wherein the probability of occurrence is expressed as a percentage of the base value occurrences in the group genome.

6. The method of claim 1, wherein the base value is one of adenine, cytosine, guanine and thymine.

7. The method of claim 6, further comprising the step of:
determining the probability of occurrence for at least three of adenine, cytosine, guanine and thymine in the reference sequence.

8. The method of claim 7, further comprising:
calculating the probability of occurrence for a fourth of adenine, cytosine, guanine and thymine as the difference of 100% probability of occurrence less the sum of the probability of occurrence for the at least three of nucleotide bases adenine, cytosine, guanine, and thymine.

9. The method of claim 6, wherein the determined probability of occurrence is representative of the probability of occurrence of each of adenine, cytosine, guanine and thymine.

10. The method of claim 6, wherein the probability of occurrence of one of adenine, cytosine, guanine, and thymine is 100%.

11. A system comprising:
a memory that stores computer-readable code; and
a processor operatively coupled to the memory, the processor configured to implement the computer-readable code, the computer-readable code configured to:
determine a probability of occurrence for a base value in a reference sequence based on base value occurrences in the group genome; and
insert the determined probability of occurrence in the reference sequence.

12. The system of claim 11, wherein the probability of occurrence is determined for a plurality of base values in the reference sequence.

13. The system of claim 11, wherein the probability of occurrence is expressed as a percentage of the base value occurrences in the group genome.

14. The system of claim 11, wherein the base value is one of adenine, cytosine, guanine and thymine.

15. An article of manufacture comprising:
a computer-readable medium having computer-readable code embodied thereon, the computer-readable code comprising:
a step to determine a probability of occurrence for a base value in a reference sequence based on base value occurrences in the group genome; and
a step to insert the determined probability of occurrence in the reference sequence.

16. The article of manufacture of claim 15, wherein the probability of occurrence is determined for a plurality of base values in the reference sequence.

17. The article of manufacture of claim 15, wherein the probability of occurrence is expressed as a percentage of the base value occurrences in the group genome.

18. The article of manufacture of claim 15, wherein the base value is one of adenine, cytosine, guanine and thymine.
